(19) United States
(12) Patent Application Publication
(10) Pub. No.: US 2026/0331265 A1
(43) Pub. Date: Oct. 22, 2026

(54) METHOD AND SYSTEM FOR TRAINING IMAGE CLASSIFICATION MODEL FOR MULTI-LABEL IMAGES, AND METHOD FOR CLASSIFYING IMAGES THROUGH IMAGE CLASSIFICATION MODEL

(71) Applicant: LG MANAGEMENT DEVELOPMENT INSTITUTE CO., LTD., Seoul (KR)

(72) Inventor: Jong Seong JANG, Seoul (KR)

(21) Appl. No.: 19/730,076

(22) Filed: Jul. 2, 2026

Related U.S. Application Data
(63) Continuation of application No. 19/211,349, filed on May 19, 2025.
Application No. 19/211,349 is a continuation of application No. PCT/KR2023/018499, filed on Nov. 16, 2023.

(30) Foreign Application Priority Data
Nov. 18, 2022 (KR) 10-2022-0155020

(51) Int. Cl. G16H 30/40; G06V 10/74; G06V 10/774; G06V 10/82; G06V 30/416; G16H 15/00

(52) U.S. Cl. CPC G16H 30/40; G06V 10/761; G06V 10/774; G06V 10/82; G06V 30/416; G16H 15/00

(57) ABSTRACT

A method and a computing system including a memory and a processor may train an image classification model and may perform operations comprising: augmenting text data in sample data of a plurality of image-text pairs; training the image classification model by fine-tuning a pretrained vision-language model on the basis of the data-augmented texts and images; and providing the trained image classification model.

### CROSS-REFERENCE TO RELATED APPLICATIONS

This application is a continuation of U.S. application Ser. No. 19/211,349, filed on May 19, 2025, which is a continuation of International Application No. PCT/KR2023/018499, filed on Nov. 16, 2023, which claims priority from and the benefit of Korean Patent Application No. 10-2022-0155020, filed on Nov. 18, 2022, which are all hereby incorporated by reference in their entireties.

### TECHNICAL FIELD

The present disclosure generally relates to a machine learning model related technology including a neural network. More specifically, some embodiments of the present disclosure may relate to a method and a system for training an image classification model for multi-label images, and a method and a system for classifying images through the image classification model.

### BACKGROUND

Techniques for pathology classification of medical images with deep neural networks have been developed in various areas.

However, it is difficult to construct a large-scale, high-quality training data set for robustly training a deep neural network because a labeling task for a medical image is required for training an existing deep neural network, and deep medical knowledge is required for labelers.

However, with recent emergence of a pre-trained deep neural network for large-scale general domain data, solutions to the problems have been proposed using a deep neural network pretrained with general domain data.

For example, contrastive language-Image pretraining (CLIP) proposes a contrastive pretraining scheme for self-supervised learning of a general image-text pair, and dramatically improves a pretraining performance of a visual transformer.

Based on Alec Radford et al., Learning Transferable Visual Models From Natural Language Supervision (Feb. 26, 2021) (hereinafter “Related Art Document 1”), Ekin Tiu et al, CheXzero: Expert-level detection of pathologies from unannotated chest x-ray images via self-supervised learning (2022) (hereinafter “Related Art Document 2”) has developed a model for classifying the pathology of a chest x-ray image by fine-tuning a chest x-ray image and a pathology annotation thereto into a pair of contrastive image-texts using a contrastive image-text encoder.

Related Art Document 2 fine-tunes the pretrained visual transformer of Related Art Document 1 with more than 200,000 pairs of chest X-ray images and diagnostic reports for each X-ray image, and performs a zero-shot pathology analysis with the fine-tuned model, showing a diagnostic accuracy level similar to that of an authorized radiologist. Related Art Document 1 shows that pathology classification can be performed with a minimal training data set without a labeled image-text pair with a medical image, which is an existing training data set.

Zifeng Wang et al., MedCLIP: Contrastive learning from unpaired medical images and text (2022), (hereinafter “Related Art Document 3”) for another pathology classification model partially deals with the problem of multi-labeling for images, but as a method for solving this problem, fine tuning is performed on a medical image through an expert's labeled training data set.

On the other hand, a research to augmenting limited training data is also active.

For data augmentation, sample data may be added in such a scheme that the image is subjected to various random deformations (e.g., cropping, horizontal flipping, affine transform, color jittering, and Gaussian blurring) and then matched again with an existing text. In this case, metadata may be further used. For example, Yen Nhi Truong Vu et al., Contrastive learning leveraging patient metadata improves representations for chest x-ray interpretation (2021) (Machine Learning for Healthcare Conference) (hereinafter “Related Art Document 5”) (MedAug) discloses a technology of performing training with a similar positive pair of images including the same study ID, and Shekoofeh Azizi et al., Big self-supervised models advance medical image classification (2021) (IEEE/CVF International Conference on Computer Vision) (hereinafter “Related Art Document 6”) (MICLe) discloses a technology of fine-tuning by identifying an image of the same person using metadata and giving a weight during pretraining. Prior Art Documents 5 and 6 described above disclose a method in which, in a case where an image for the same study ID and the same person is insufficient, the image is modified to augment data, and the augmented data is trained to be a positive pair.

Maria de la Iglesia-Vay'a. Padchest: A large chest x-ray image dataset with multi-label annotated reports. Medical image analysis (2020) (hereinafter “Related Art Document 7) (MCL: Masked Contrastive Learning) discloses a training scheme having a new loss function so that data augmentation or similarity of data determined as a positive pair on metadata is high.

### RELATED ART DOCUMENTS

Related Art Document 1: Alec Radford, Jong Wook Kim, Chris Hallacy, Aditya Ramesh, Gabriel Goh, Sandhini Agarwal, Girish Sastry, Amanda Askell, Pamela Mishkin, Jack Clark, Gretchen Krueger, Ilya Sutskever. Learning Transferable Visual Models From Natural Language Supervision, (Feb. 26, 2021)

Related Art Document 2: Ekin Tiu, Ellie Talius, Pujan Patel, Curtis P Langlotz, Andrew Y Ng, and Pranav Rajpurkar. Expert-level detection of pathologies from unannotated chest x-ray images via selfsupervised learning. Nature Biomedical Engineering (2022)

Related Art Document 3: Zifeng Wang, Zhenbang Wu, Dinesh Agarwal, and Jimeng Sun. Medclip: Contrastive learning from unpaired medical images and text (2022) (EMNLP)

Related Art Document 4: Yuhao Zhang, Hang Jiang, Yasuhide Miura, Christopher D Manning, and Curtis P Langlotz. Contrastive learning of medical visual representations from paired images and text. arXiv preprint arXiv: 2010.00747 (2020).

Related Art Document 5: Yen Nhi Truong Vu, Richard Wang, Niranjan Balachandar, Can Liu, Andrew Y Ng, and Pranav Rajpurkar. Medaug: Contrastive learning leveraging patient metadata improves representations for chest x-ray interpretation (2021) (Machine Learning for Healthcare Conference)

Related Art Document 6: Shekoofeh Azizi, Basil Mustafa, Fiona Ryan, Zachary Beaver, Jan Freyberg, Jonathan Deaton, Aaron Loh, Alan Karthikesalingam, Simon Kornblith, Ting Chen, et al. Big self-supervised models advance medical image classification (2021) (IEEE/CVF International Conference on Computer Vision)

Related Art Document 7: Maria de la Iglesia-Vay'a. Padchest: A large chest x-ray image dataset with multi-label annotated reports. Medical image analysis, 66:101797 (2020)

### SUMMARY

Some embodiments of the present disclosure may provide an image classification model that accurately performs each label classification for a multi-label nature by fine-tuning a vision language model (e.g., CLIP) pre-trained with contrastive supervising.

Further, certain embodiments of the present disclosure may provide an image classification method that may perform classification for each of multiple labels for images of a category having multi-label nature by using an image classification model.

For example, some embodiments of the present disclosure may provide a pathology classification method in which an image classification model performs diagnosis for a plurality of pathologies of a medical image.

In particular, according to certain exemplary embodiments of the present disclosure, a method for contrastively training multi-label images may provide a new fine tuning technique that may classify pathologies by zero shots by considering a multi-label nature of a medical image text pair.

Specifically, a method for contrastively training multi-label images according to some exemplary embodiments of the present disclosure may provide a new fine-tuning strategy based on sentence sampling and positive-pair loss mitigation to improve the self-stream zero-shot pathology classification performance.

According to an embodiment of the present disclosure, a method for training image classification model by a computing system including a memory and a processor includes: augmenting text data in sample data of a plurality of image-text pairs; training the image classification model by fine-turning a pretrained vision-language model based on the data-augmented texts and an image; and providing the trained image classification model.

According to an embodiment of the present disclosure, a computing device for individually classifying a plurality of pathologies for an input medical image having a multi-label nature includes: a memory storing an image classification model classifying a plurality of pathologies for the input medical image; and one or more processors operatively combining the image classification mode and the data of the memory, and
the one or more processors obtain raw data composed of a plurality of multi-label images, and reports including descriptions of the multi-label images, detect a multi-label image according to a preset criterion in the obtained raw data, detect a report associated with the detected multi-label image, individually separating sentences included in the plurality of reports to detect n sentences, augment text data by generating a plurality of active directories based on the n detected sentences, train an image classification model by fine-turning a vision-language model pretrained based on the data-augmented texts and an image, and provide the trained image classification model.

A method for training an image classification model for a multi-label image according to some embodiments of the present disclosure can provide an image classification model with improved downstream zero-shot performance by fine-tuning an image classification model pretrained with a general domain image-text pair into a training data set without a separate labeling task.

An image classification model and An image classification method using the same according to certain embodiments of the present disclosure can accurately perform redundant classification by determining whether each label is applied to an image having a multi-label nature.

Specifically, when a pre-trained vision-language model is fine-turned by an image classification training method according to an exemplary embodiment of the present disclosure, the AUROC is improved by 5.77% as a result of performing zero-shot pathology classification on average. In addition, an image classification model according to certain of the present invention shows a performance much similar to or better than that of a radiologist certified by a committee.

Specifically, the image classification model according to an exemplary embodiment of the present disclosure performs image label classification with relatively high accuracy in the zero-shot classification for five major diseases of a CheXpert dataset (F1 score: 0.619 to 0.625, MCC: 0.530 to 0.544).

### BRIEF DESCRIPTION OF THE DRAWINGS

FIG. 1 illustrates a block diagram of a computing system for training and controlling an image classification model that effectively and accurately performs image classification of multiple labels according to an embodiment of the present disclosure.

FIG. 2 illustrates a block diagram of a computing device for training and controlling an image classification model that effectively and accurately performs image classification of multiple labels according to an embodiment of the present disclosure.

FIG. 3 illustrates a block diagram in another aspect for a computing device for training and controlling an image classification model that effectively and accurately performs image classification of multiple labels according to an embodiment of the present disclosure.

FIG. 4 is a flowchart of a method for training an image classification model and a method for classifying an image using the trained image classification model according to an embodiment of the present disclosure.

FIG. 5 includes a conceptual diagram (a) illustrating a relationship between an embedding vector of a medical image and an embedding vector of a diagnosis report of the medical image, and a conceptual diagram (b) illustrating a relationship between the embedding vector of the medical image and an embedding vector of data magnified active directories from the diagnosis report of the medical image.

FIG. 6 illustrates an example illustrating a text data magnification method according to an embodiment of the present disclosure.

FIG. 7 illustrates a conceptual diagram for illustrating an example of a state in which an image classification model is trained according to an embodiment of the present disclosure.

FIG. 8 illustrates a conceptual diagram for illustrating an example of a state of classifying an image through a trained image classification model according to an embodiment of the present disclosure.

FIG. 9 illustrates an example in which an image classification model provides a result of performing classification of images of multiple labels according to an embodiment of the present disclosure.

### DETAILED DESCRIPTION OF EMBODIMENTS

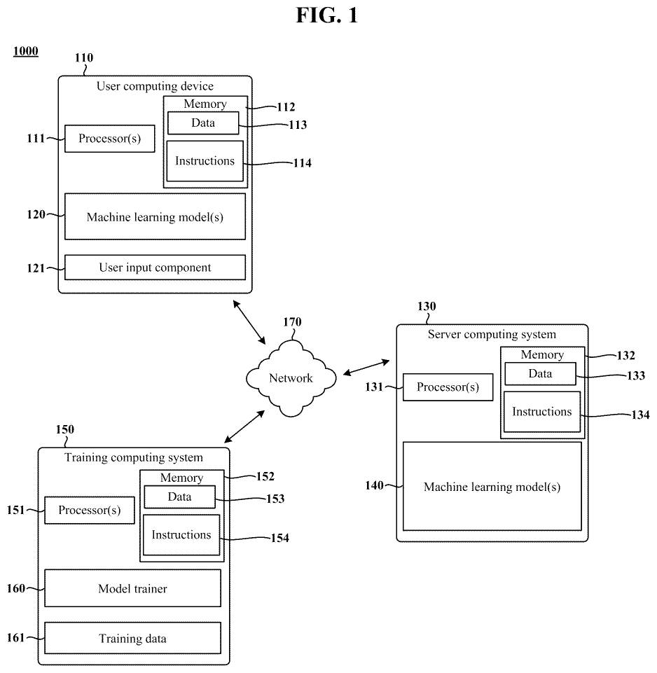

FIG. 1 illustrates a block diagram of a computing system for training and controlling an image classification model configured to perform image classification of multiple labels according to an embodiment of the present disclosure.

Examples of image types which may be used in some embodiments of the present disclosure may include a video frame, LiDAR point cloud, an X-ray image, a computer tomography scan, a hyperspectral image, and/or various other types of images, but not limited thereto.

In an embodiment, a training data 160 may comprise an image-text pair, and the image may be an image with a multi-label nature that may correspond to at least one of multi-labels, and the text may include information about whether the image corresponds to each of the multi-labels in a report that includes a description of the image.

As such, the text in the image-text pair may refer to a text detected in association with an image in sample data according to a specific criterion for self-supervised learning with sentences associated with the image. For example, the text may mean a text associated with an image among texts included in the sample data, which is not a label generated by a labeler in a labeling task on the image.

For example, a diagnosis report for a medical image may be an image-text pair of training data, and more specifically, may mean a bundle of a lung radiation image and a pathology diagnosis report prepared by an expert for a lung radiation image.

The training data 160 and image classification target data may be provided by a user computing device or a user's terminal 110 and/or a server computing system or a server 130. When the training computing device 150 trains a machine learning model 120 with respect to specific data of the user computing device 110, the machine learning model 120 may be characterized as a personalized model.

In addition, the model trainer 160 includes a computer logic utilized to provide a desired or intended function. The model trainer 160 may be implemented as hardware, firmware, and/or software controlling a universal processor. For example, in an embodiment, the model trainer 160 may include a program file stored in a storage device, and may be loaded to the memory 152 and executed by one or more processors 151.

A network 170 includes, for instance, a 3rd Generation Partnership Project (3GPP) network, a Long Term Evolution (LTE) network, a World Interoperability for Microwave Access (WIMAX) network, Internet, a Local Area Network (LAN), Wireless Local Area Network (LAN), a Wide Area Network (WAN), a Personal Area Network (PAN), a Bluetooth network, a satellite broadcasting network, an analog broadcasting network, and/or a Digital Multimedia Broadcasting (DMB) network, etc., but is not limited thereto.

In general, communication through the network 170 may be performed through various communication protocols (e.g., TCP/IP, HTTP, SMTP, and FTP), encoding or formats (e.g., HTML and XML, etc.), and/or protective schemas (e.g., VPN, secure HTTP, and SSL) by using uses any type of wired and/or wireless connection.

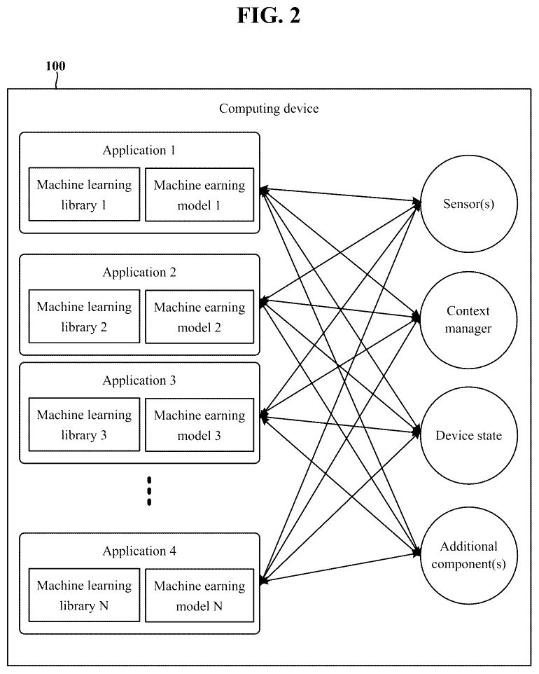

FIG. 2 illustrates a block diagram of a computing device for training and controlling an image classification model configured to perform image classification of multiple labels according to an embodiment of the present disclosure.

Referring to FIG. 2, a computing device 100, which may be included in the user computing device 110, the server computing system 130, and/or the training computing system 150, includes multiple applications (e.g., applications 1 to N). Each application may include a machine learning library and one or more machine learning models. For example, the application may include an application for an image processing (e.g., detection, classification, segmentation, etc.), a text messaging application, an email application, a dictation application, a virtual keyboard application, a browser application, a chat-bot application, and so on.

In an embodiment, the computing device 100 may include a model trainer 160 configured to train an image classification model, and may store and operate the trained image classification model to perform image classification for an input image.

Each application of the computing device 100 may communicate or be operably associated with, for example, multiple other components of the computing device 100, such as one or more sensors, context managers, device state components, and/or additional components. In an embodiment, each application may communicate with each device component by using an API (e.g., a public API). In an embodiment, the API used by each application may be specific for the corresponding application.

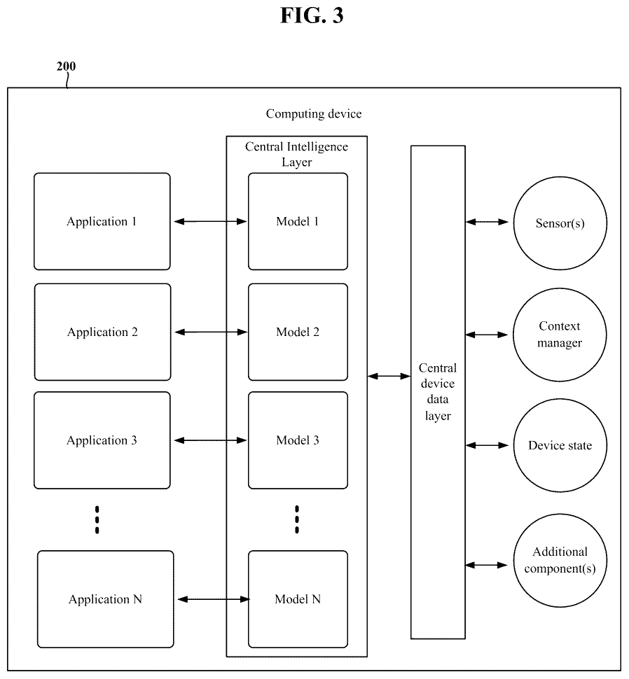

FIG. 3 illustrates a block diagram in another aspect for a computing device 100 for training and controlling an image classification model configured to perform image classification of multiple labels according to an embodiment of the present disclosure.

Referring to FIG. 3, the computing device 100 includes multiple applications (e.g., applications 1 to N). Each application may communicate with a central intelligence layer. For example, the applications may include an image processing application, a text message application, an email application, a dictation application, a virtual keyboard application, a browser application, etc. In an embodiment, each application may communicate with the central intelligence layer (and a model stored therein) by using an API (e.g., a common API for all applications).

The central intelligence layer may include multiple machine learning models. For example, as illustrated in FIG. 3, one or at least some of the machine learning models may be provided for each application, and managed by the central intelligence layer. In another implementation example, two or more applications may share a single machine learning model. For example, in some implementation examples, the central intelligence layer may provide the single model for all applications. In some implementation examples, the central intelligence layer may be included in an operating system of the computing device 100 or implemented differently therefrom.

The central intelligence layer may communicate with a central device data layer. The central device data layer may be a centralized data storage for the computing device 100. As illustrated in FIG. 3, the central device data layer may communicate with multiple other components of the computing device 100, such as one or more sensors, context managers, device state components, and/or additional components, but not limited thereto. In some implementation examples, the central device data layer may communicate with each device component by using an API (e.g., a private API).

The techniques describe in the present disclosure may refer to information sent from servers, databases, software applications and other computer-based systems, as well as taken action and information transmitted to the system or transmitted from the system. An intrinsic flexibility of the computer-based systems will perceive that extensive possible components, combinations, division of combinations and tasks and a functionality between components and from the components are permitted. For example, some processes described in certain embodiments of the present disclosure may be implemented by using a single device or component or multiple devices or components which operate as combinations. Databases and applications may be implemented in a single system or components distributed over multiple systems. The distributed components may operate in sequence or in parallel.

### [Image Classification Model Training Method and Image Classification Method Using Image Classification Model]

Hereinafter, a method in which a computing system 1000 configured to train and/or operate an image classification model trains the image classification model and performs image classification through the trained image classification model according to certain embodiments of the present disclosure will be described in detail.

Here, an image to be classified may be an image having a multi-label nature in which it is individually determined whether applications are individually applied to a plurality of labels, respectively. For example, a medical image (e.g., a chest X-ray examination image) including an X-ray image, a computed tomography scan image, and the like may be used to diagnose for a plurality of pathologies (e.g., lung opacity, edema, and pneumonia), respectively, so that a plurality of pathologies that may be diagnosed for the medical image may correspond to a plurality of labels.

In the following description, the medical image is represented and described as an image having the multi-label nature, and a label for the medical image is described with reference to diagnostic pathologies for the medical image.

The computing system 1000 according to an embodiment of the present disclosure trains the image classification model by fine-tuning a pre-trained vision-language model through sample data (for example, image-text pair) for the medical image, effectively training the image classification model capable of accurately classifying the medical image of the multi-label nature, resulting in a consistent improvement in the multiple pathology classification for the zero-shot medical image, and may perform the pathology classification with better accuracy than the certified radiologist of the committee.

Here, the pre-trained vision-language model may be contrastive trained models from image-text pairs in the general domain, such as contrastive language-image pretraining (CLIP), BigGAN, self-supervision meets language-image pretraining (Slip) or/and unified framework for contrastive language-image pretraining (Uniclip).

The fine tuning technique in the image classification model training method in an embodiment of the present disclosure May 1) perform data augmentation to solve a problem of insufficient sample data. In this way, a labeling task (e.g., an annotation task) for the medical image may not be required, and fine tuning of the image classification model may be smoothly performed by extracting only texts required for the pathology classification for the medical image, and augmenting the extracted texts to obtain multiple sample data.

In addition, the fine tuning technique in the image classification model training method in an embodiment of the present disclosure may further improve classification accuracy for each label for the medical image having a multi-label nature by 2) applying a relaxing loss function that relaxes a vector similarity between positive pairs of image-texts.

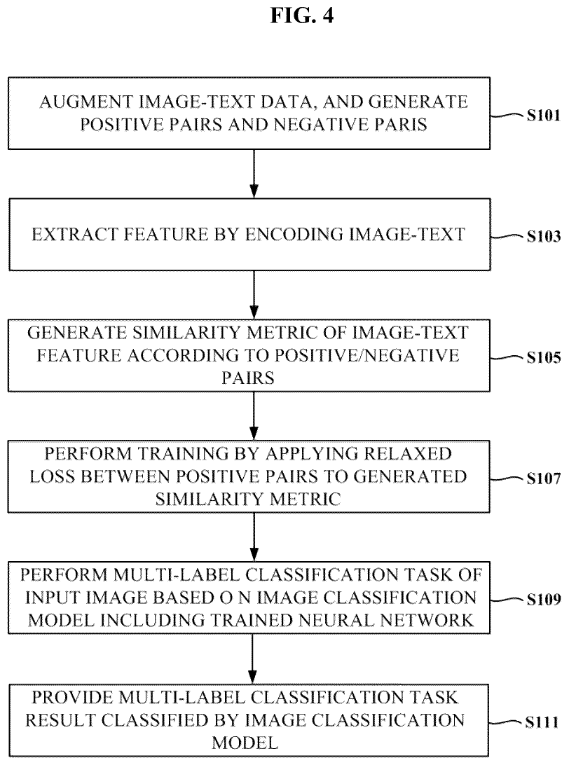

Hereinafter, some exemplary embodiments of an image classification model training method will be described in more detail with reference to FIGS. 4 to 7.

The image classification model training method according to the embodiment may include one or more of a step S101 of generating sample data, a step S103 of encoding each image-text of the sample data to output an embedding, a step S105 of generating a similarity metric between an output image embedding and a text embedding as a positive or negative pair, and a step S107 of training the positive pair to have a similarity based on a loss function obtained by relaxing the generated similarity metric. In addition, the image classification method based on the image classification model may include a step S109 of performing a classification task for whether the input image corresponds to each of the multiple labels based on the image classification model trained as described above, and a step S111 of providing a result of the multi-label classification task for the input image according to the image classification task.

At step S101, the computing system 1000 may generate the sample data.

For instance, the computing system 1000 may detect a medical image corresponding to a predetermined criterion in raw-data, and detect a diagnosis report associated with the detected medical image. Here, the raw data may be an electronic medical record (EMR) including multiple medical images and a diagnosis report for the medical images.

For example, the computing system 1000 May 1) determine a specific body organ in the medical image, and 2) when determining an image type (e.g., an RGB image, a radiation image, a computed tomography image, etc.), detect a specific type of the image for the specific body organ set through the image detection model as an image of the sample data.

The computing system 1000 may then detect a diagnosis report for the detected medical image of the sample data. For example, the computing system 1000 may determine an associated diagnosis report as the text of the sample data based on metadata or/and a creation template, etc., according to a creation format of the raw data. For example, when the computing system 1000 sets a criterion according to the raw-data creation format in a scheme of extracting a sentence including an ID matching an ID of the medical image and/or a scheme of extracting a sentence described at a front end or a rear end of the medical image, the computing system 1000 may extract the associated diagnosis report as the sample data according to the set criterion. In this case, the computing system 1000 may detect the diagnosis report to include both a paragraph for “findings” and a paragraph for “impression” that contain information in the pathology classification for the medical image.

The computing system 1000 may then match the medical image of the sample data thus extracted with the diagnosis report and store the matched medical image and the diagnosis report as the image-text pair of the sample data.

In addition, the computing system 1000 may perform data augmentation in order to overcome the lack of the sample data that is relatively small compared to the training data of the pre-trained vision-language model. Here, the data augmentation may mean that additional sample data is generated and augmented based on existing sample data.

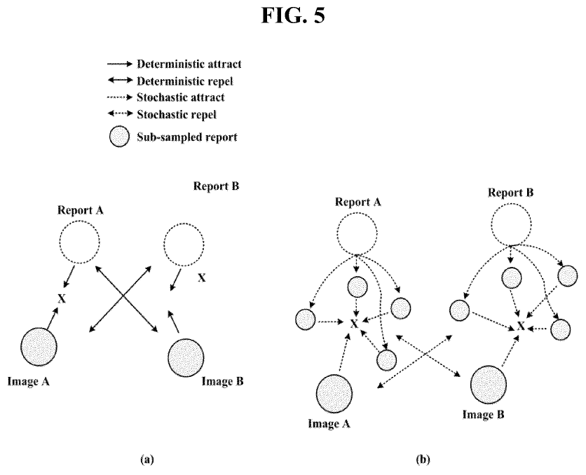

Referring to FIG. 5, when an entire diagnosis report associated with the medical image is set as a text and set as an image-text pair, (a) a matching rate of the similarity between the image and the text becomes too high, such as a diagram, and becomes a negative pair for the diagnosis report for another image that is not associated with the image but has shareable information, and is not suitable for multi-label classification. Relatively, when a sub-report including information for each label is additionally generated for the diagnosis report for the image as in (b) of FIG. 5 to augment the data, it is possible to perform training so that a similarity may be matched between the medical image and the sub-report not associated with the medical, and thus texts suitable for learning the medical image of the multi-label nature may be augmented.

Details of a specific exemplary method of the data augmentation according to an embodiment will be described.

The data augmentation according to an embodiment may not be performed for the medical image. In general, schemes such as random cropping, rotation, color conversion, viewpoint change, blurring, random noise addition, etc., which are image augmentation techniques may distort critical meanings in detecting pathologies from medical images.

The data augmentation at step S101 of FIG. 4 according to an embodiment may be random text augmentation performed only for the text.

Specifically, the random text data augmentation method may perform a task of extracting only a paragraph related to the pathology classification from the diagnostic report, thereby removing unnecessary tokens.

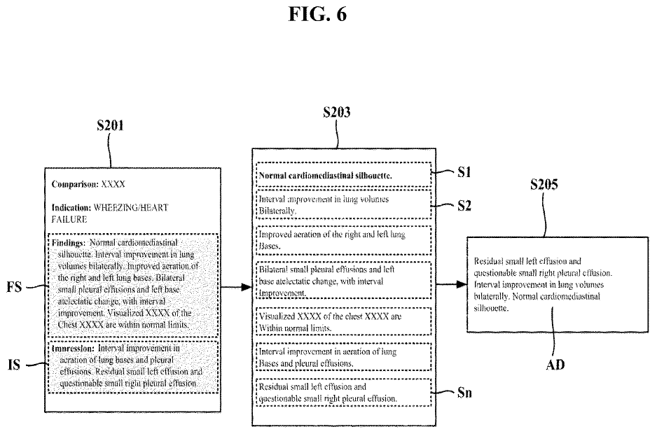

Referring to FIG. 6, at step S201, the data augmentation method may reconstruct the diagnosis report by extracting a paragraph corresponding to a predetermined table of contents (e.g., “findings” and “impression”). A findings paragraph FS and an impression paragraph IS in the diagnosis report comprise meaningful contents for a pathology classification, and some of the sentences are composed of short and shaping, so the findings paragraph FS and the impression paragraph IS may be text sample data suitable for the fine tuning.

Then, at step S203, the data augmentation method according to an embodiment of the present disclosure may separate each of the sentences S1, S2, . . . , and Sn included in the reconstructed diagnosis report into n sentences. Thereafter, at step S205, in the data augmentation method according to an embodiment of the present disclosure, a plurality of texts are additionally generated based on n separated sentences S1, S2, . . . , and Sn to augment the text data.

In an embodiment, when the number of separated sentences S1, S2, . . . , and Sn in the diagnosis report matched with a first medical image is n, and the text includes m sentences (n>m), n separated sentences S1, S2, . . . , and Sn are randomly sampled and combined to generate nCm sentences as an active directory AD for the first medical image. Here, the number of sentences of the active directory AD may be fixed to m, and the number of sub-sentences may be determined differently or variably according to the number of separated sentences of each diagnosis report. In another embodiment, a scheme of randomly determining the number of sentences and randomly sampling the number of sentences of the active directory AD from the separated sentences of the diagnosis report may be taken.

Through the text augmentation method, it is possible to additionally generate multiple active directories ADs that may be positive pairs for the first medical image, and the active directories ADs having the multiple positive pairs each express a rich meaning for a specific pathology, so that a rich meaning may be learned for the medical image having the multi-label nature. Further, for other second medical images with overlapped pathologies (labels), negative natures of the active directories ADs may be reduced to train to locate embeddings of active directories ADs with similar pathologies and the second medical image close to each other.

After the text data augmentation is performed, a plurality of active directories ADs are additionally generated for the medical image to enlarge the sample data, so that the sample data may increase in proportion to the additionally generated text data.

Thereafter, the computing system 1000 may map the sample data for the text data augmented for the diagnosis report of the medical image in the positive pair and map the sample data in the negative pair with the other medical image to sufficiently augment positive sample data and negative sample data.

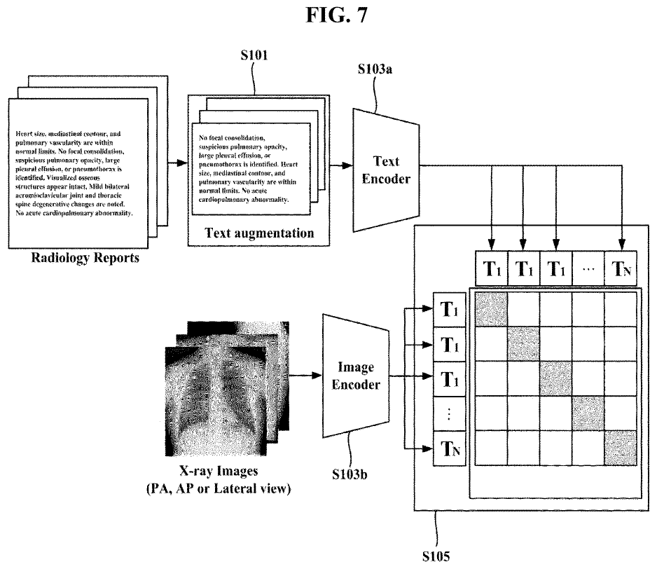

In addition, referring to FIG. 7, at steps S103a and S103b, the computing system 1000 may control each of the medical images of the sample data to output image embedding vectors (hereinafter, referred to as “embeddings”) representing an image feature through an image encoder, and control active directories to output text embedding vectors (hereinafter, referred to as “embeddings”) representing a text feature through a text encoder.

Next, at step S105, the computing system 1000 may classify sample data according to a batch size, and match the mapped positive pair and negative pair for the sample data for each classified batch into a positive pair or a negative pair of the image embedding and the text embedding to generate a similarity metric.

Here, the similarity may mean a dot product between the image embedding and the text embedding.

That is, the similarity metric may be a matrix that lists embedding samples of the positive pair and embedding samples of the negative pair such that rows and columns of the positive pair match.

Thereafter, the computing system 1000 may perform training according to the loss function to cause a spatial distance between the positive embedding samples to decrease and cause a spatial distance between the negative embedding samples to increase.

That is, contrastive learning may be performed by defining the loss function so as to achieve

$$\frac{score ( f ( x ) , f ( {x}^{+} ) )}{Positive samples} >> \frac{score ( f ( x ) , f ( {x}^{-} ) )}{Negative samples} .$$

For example, the computing system 1000 may perform training by applying InfoNCE Loss which is a first loss function (Equation 1) to the similarity metric.

$${l}_{i}^{v \to u} = - log \frac{exp ( sim ( {v}_{i} , {u}_{i} ) / τ )}{{\sum }_{k = 1}^{N} exp ( sim ( {v}_{i} , {u}_{k} ) / τ )} \qquad [ Example 1 ]$$

Where v and u represent normalized embedding vectors of an image and a text, respectively, (vi, uᵢ) represents a positive embedding sample and (vᵢ, uₖ) represents a negative embedding sample, sim represents a function of calculating the similarity between the embedding vectors, which may be, for example, a cosine similarity, t represents a learnable temperature, and N represents a batch size.

That is, an image i is paired with one positive text and N−1 negative texts to construct the similarity metric. For lv→u, a degree of loss may be learned through the first loss function such that the text i has a high similarity to the image i.

However, when the first loss function is applied as it is to learn the similarity of the positive pair to be completely high and the similarity of the negative pair to be completely low in the similarity metric, it may be difficult to learn a similarity between positive texts for the image i to be high.

In order to relax this problem, the computing system 1000 may take a scheme of calculating and learning the similarity of the similarity metric according to a second loss function obtained by modifying the function sim for calculating the similarity.

$$sim ( {v}_{i} , {u}_{i} ) = \{ \begin{matrix}\frac{1}{1 + exp ( - α ( {v}_{i} \cdot {u}_{j} - t ) )} , & \begin{matrix}if i = j and \\ {v}_{i} \cdot {u}_{j} \geq t\end{matrix} \\ {v}_{i} \cdot {u}_{j} / 2 t , & \begin{matrix}else if i = j and \\ t > {v}_{i} \cdot {u}_{j} \geq 0\end{matrix} \\ {v}_{i} \cdot {u}_{j} , & otherwise\end{matrix} \qquad [ Example 2 ]$$

Where vᵢ*uⱼ represents a cosine similarity between the image embedding vectors and the text embedding vectors, α represents a slope coefficient of a sigmoid function, and t (0<t<1) represents the threshold value of the similarity which regards the level of similarity between the sample data as “positive”.

That is, when the similarity is greater than t, the similarity may be calculated to quickly approach 1.

In this way, the second loss function, which may perform calculation to relax the matching between the image and the text of the positive pair, may make room for the matching with respect to the image even for texts of the sample that are not positively mapped, thereby training an image classification model in which a plurality of labels are accurately classified into images having the multi-label nature.

In this way, the computing system 1000 may train the image classification model by using two fine tuning techniques described below for a pretrained vision-language model, so that 1) sample data may be sufficiently secured through text data augmentation that is not distorted and has a rich meaning for each pathology, and 2) a loss function in which the matching of positive pairs between sample data is relaxed is applied and leaned to the similarity metric to train the image classification model that accurately classifies whether labels are applied redundantly to medical images having the multi-label nature.

In addition, at step 109, the computing system 1000 may perform a task of classifying multiple labels for the input image through the image classification model trained as such.

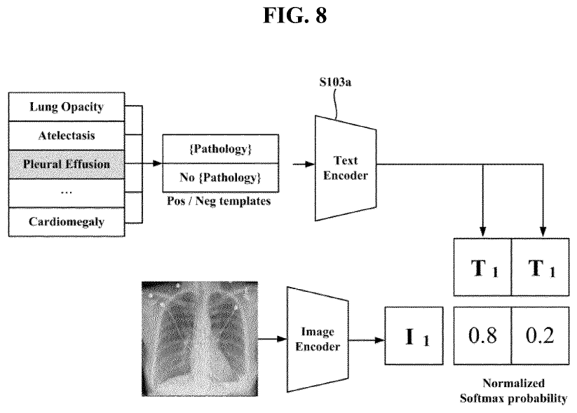

Referring to FIG. 8, the computing system 1000 may perform prompt engineering to perform classification of each multi-label for an input image.

In detail, the computing system 1000 may define multiple labels for the medical image. For example, each of the pathologies defined in the training data set for the fine tuning may be determined as the label. In an embodiment, the computing system 1000 may input active directories generated by augmenting the text data of the diagnostic report for the medical image into a natural language analysis model to extract detectable pathologies, and determine the extracted pathologies as the labels.

The computing system 1000 may then input each of the labels into a positive template or a negative template for the input image to generate a sentence for determining whether to classify the labels.

For example, in order to confirm whether a label for a lung consolidation is classified, the computing system 1000 may generate a positive sentence including a lung consolidation positive and a negative sentence including a lung consolidation negative.

Then, the computing system 1000 may input the positive sentence and the negative sentence as texts into the text encoder of the pretrained image classification model to output the text embedding for the positive sentence and the text embedding of the negative sentence, and input the input image to the image encoder of the pretrained image classification model to output the image embedding.

Then, the computing system 1000 calculates an image embedding-normalized SoftMax similarity (i.e., a distance between respective vectors) for each of the text embedding of the positive sentence and the text embedding of the negative sentence, and calculates values of a classification positive similarity and a negative similarity for the corresponding pathology, and classifies the input image into the corresponding pathology when the similarity to the positive is higher, and does not classify the input image into the corresponding pathology when the similarity to the negative is higher.

As such, the computing system 1000 may generate a positive sentence and a negative sentence for each of the multiple labels according to the template, and input the text including the generated positive sentence and negative sentence and the input image into the image classification model to determine whether to classify each label.

Through such prompt engineering, the computing system 1000 may redundantly determine image classifications for multiple labels.

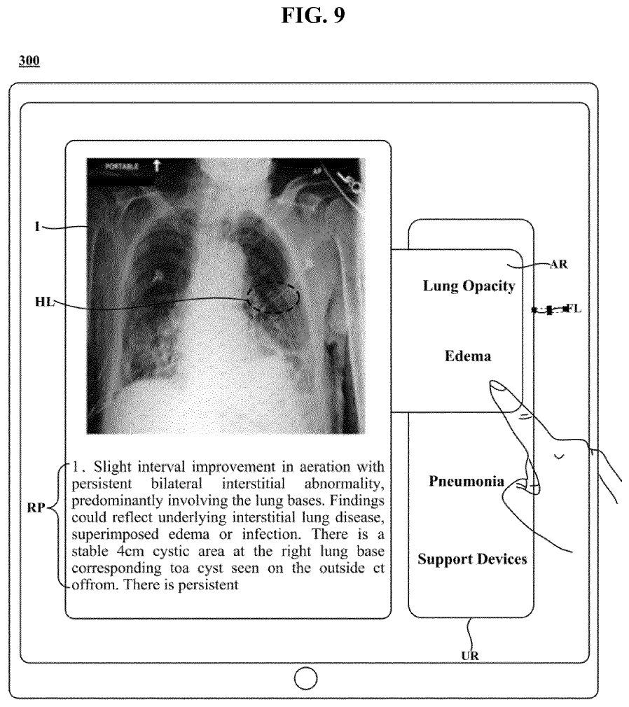

Referring to FIG. 9, the computing system 1000 may display a classified label AR by classifying whether an input image I corresponds to one or more labels (pathologies) as described above, and in this case, provide unclassified pathologies UR to be displayed together to allow an user to confirm a label FL determined by an image classification model. Through this, the user may intuitively grasp a pathology FL which the image classification model may determine for the medical image, and assist in performing efficient diagnosis of the medical image.

In addition, the computing system 1000 may restore and provide a diagnosis report RP based on a text embedding having a high similarity according to whether to apply multiple labels. That is, the computing system 1000 may also generate and provide an artificial intelligence diagnosis report RP for the input image I through an image classification model trained from active directories generated through text augmentation for the diagnosis report.

In addition, the computing system 1000 may calculate an association between at least a partial region of the input image I and the classified label, and highlight (HL) and provide a region of the input image I having a high similarity with the classified label, thereby additionally assisting a diagnosis of a user.

Hereinafter, three image classification models are trained by performing fine tuning according to an embodiment of the present disclosure for three example models among the pretrained vision-language models through the contrastive training, and a zero-shot performance evaluation is performed for each of three image classification models and the existing preceding image classification models (CheXzero and MedCLIP) so as to prove an effect.

Here, a first image classification model fine-tuned with the example model may be a model fine-tuning a contrastive language-Image pretraining (CLIP)-based framework, a second image classification model may be a model fine-tuning an self-supervision meets language-image pretraining (Slip) based framework, and a third image classification model may be a model fine tuning a unified framework for contrastive language-image pretraining (Uniclip) based framework.

TABLE 1

| | | # of | # of | # of |
|---|---|---|---|---|
| | | Images | Reports | classes |
| Fine-tuning | MIMIC-CXR [12] | 377,110 | 227,835 | — |
| | CheXpert [symbol] [11] | 234 | 200 | — |
| Evaluation | CheXpert [symbol] [11] | 668 | 500 | 14 |
| | Open- [symbol] [1] | 7,470 | 3,955 | 14 |
| | P [symbol] Chest [3] | 39,053 | 26,413 | 61 |
| | VinD [symbol] -CXR [21] | 3,000 | — | 20 |

[symbol]

indicates data missing or illegible when filed

Table 1 shows a data set used for fine tuning and a data set used for evaluating the zero-shot performance according to the embodiment.

In an embodiment, the training data set used for the fine tuning according to an embodiment of the present disclosure is MIMIC-CXR.

In addition, a data set for verification of CheXpert, a data set of PadChest, an Open-i chest X-ray data set, and VinDr-CXR are used as test data sets.

Results of performing the zero-shot performance evaluation by utilizing the test data sets for the first image classification model, the second image classification model, a third image classification model, and a first preceding image classification model and a second preceding image classification model are shown in Table 2 below.

TABLE 2

| | CheXpert | | Open-i | | PadChest | VinDr-CXR |
|---|---|---|---|---|---|---|
| | avg. 5 AUC | avg. total AUC | avg. 5 AUC | avg. total AUC | avg. total AUC | avg. total AUC |
| CLIP | 0.870 ± 0.006 | 0.771 ± 0.001 | 0.785 ± 0.030 | 0.691 ± 0.010 | 0.685 ± 0.026 | 0.801 ± 0.020 |
| CLIP w/ours | 0.885 ± 0.007 | 0.789 ± 0.017 | 0.808 ± 0.008 | 0.708 ± 0.013 | 0.725 ± 0.014 | 0.842 ± 0.007 |
| SLIP | 0.831 ± 0.008 | 0.722 ± 0.008 | 0.760 ± 0.008 | 0.666 ± 0.004 | 0.684 ± 0.004 | 0.799 ± 0.018 |
| SLIP w/ours | 0.884 ± 0.009 | 0.779 ± 0.020 | 0.810 ± 0.016 | 0.719 ± 0.018 | 0.741 ± 0.005 | 0.831 ± 0.009 |
| UniCLIP | 0.851 ± 0.008 | 0.741 ± 0.037 | 0.744 ± 0.032 | 0.643 ± 0.027 | 0.684 ± 0.009 | 0.759 ± 0.019 |
| UniCLIP w/ours | 0.888 ± 0.006 | 0.802 ± 0.018 | 0.788 ± 0.016 | 0.694 ± 0.006 | 0.717 ± 0.011 | 0.843 ± 0.016 |
| CheXzero | 0.844 ± 0.008 | 0.733 ± 0.008 | 0.771 ± 0.006 | 0.684 ± 0.006 | 0.702 ± 0.011 | 0.771 ± 0.024 |
| MedCLIP* | 0.880 | 0.829 | 0.794 | 0.728 | 0.728 | 0.829 |

*use label information defined in CheXpert for training

Table 2 shows that the fine tuning technique according to an embodiment of the present disclosure continuously improves the zero-shot pathology classification performance through an average of 5.77% increase in area under ROC curve (AUROC) according to whether the first to third image classification models are subjected to fine tuning, thereby demonstrating a superiority of the fine tuning technique.

TABLE 3

| | Text | Relax | | | | | Pleural | avg. | avg. |
|---|---|---|---|---|---|---|---|---|---|
| | aug. | loss | Atelectasis | Cardiomegaly | Consolidation | Edema | Effusion | 5 AUC | total AUC |
| CLIP | | | 0.828 ± 0.016 | 0.845 ± 0.012 | 0.895 ± 0.007 | 0.861 ± 0.016 | 0.878 ± 0.020 | 0.861 ± 0.002 | 0.787 ± 0.044 |
| | | ✓ | 0.858 ± 0.009 | 0.815 ± 0.013 | 0.889 ± 0.023 | 0.875 ± 0.022 | 0.890 ± 0.006 | 0.865 ± 0.002 | 0.794 ± 0.045 |
| | ✓ | | 0.864 ± 0.004 | 0.867 ± 0.008 | 0.904 ± 0.015 | 0.903 ± 0.003 | 0.877 ± 0.021 | 0.883 ± 0.005 | 0.820 ± 0.001 |
| | ✓ | ✓ | 0.859 ± 0.006 | 0.840 ± 0.016 | 0.913 ± 0.010 | 0.881 ± 0.009 | 0.869 ± 0.012 | 0.872 ± 0.005 | 0.824 ± 0.003 |
| SLIP | | | 0.760 ± 0.046 | 0.788 ± 0.008 | 0.877 ± 0.028 | 0.847 ± 0.021 | 0.902 ± 0.010 | 0.835 ± 0.010 | 0.791 ± 0.015 |
| | | ✓ | 0.826 ± 0.010 | 0.828 ± 0.010 | 0.899 ± 0.006 | 0.873 ± 0.013 | 0.899 ± 0.010 | 0.865 ± 0.005 | 0.828 ± 0.003 |
| | ✓ | | 0.830 ± 0.022 | 0.837 ± 0.021 | 0.864 ± 0.042 | 0.899 ± 0.008 | 0.876 ± 0.013 | 0.861 ± 0.004 | 0.800 ± 0.015 |
| | ✓ | ✓ | 0.845 ± 0.014 | 0.855 ± 0.018 | 0.904 ± 0.014 | 0.905 ± 0.016 | 0.868 ± 0.009 | 0.875 ± 0.006 | 0.819 ± 0.028 |
| UniCLIP | | | 0.774 ± 0.060 | 0.833 ± 0.012 | 0.869 ± 0.016 | 0.856 ± 0.033 | 0.882 ± 0.020 | 0.843 ± 0.010 | 0.750 ± 0.016 |
| | | ✓ | 0.840 ± 0.009 | 0.818 ± 0.021 | 0.870 ± 0.012 | 0.869 ± 0.014 | 0.897 ± 0.021 | 0.859 ± 0.008 | 0.808 ± 0.023 |
| | ✓ | | 0.860 ± 0.012 | 0.862 ± 0.010 | 0.900 ± 0.019 | 0.879 ± 0.005 | 0.887 ± 0.012 | 0.877 ± 0.009 | 0.790 ± 0.020 |
| | ✓ | ✓ | 0.870 ± 0.007 | 0.844 ± 0.007 | 0.911 ± 0.002 | 0.886 ± 0.014 | 0.882 ± 0.017 | 0.878 ± 0.001 | 0.825 ± 0.028 |

* use label information defined in CheXpert for training

In addition, Table 3 shows experiments on classification accuracy by various pathologies (diseases) of the proposed method in CheXpert's test set (average±standard). For each framework, a highest performance is indicated in bold letters and a second highest performance is indicated to be underlined. Table 3 shows that the zero-shot performance is improved when the text data augmentation and the relaxation loss function are applied in the fine tuning technique.

In particular for the CheXpert data set, fine-tuning the CLIP with the image classification model training method according to an embodiment of the present disclosure sometimes shows a slightly better performance than the committee certified radiologist in detecting the five major diseases with an F1 score of 0.619 vs. 0.625 and an MCC of 0.530 vs. 0.544, which are not tabulated.

As such, some embodiments of the present disclosure may provide an image classification model including a neural network that improves downstream zero-shot performance by fine-tuning an image classification model pretrained with the general domain image-text pair without a separate labeling task.

As a result of performing zero-shot pathology classification after fine tuning four chest X-ray data sets and three pretrained models (average 5.77% AUROC increase), the image classification model according to an embodiment of the present disclosure may continuously show dramatically improved zero-shot pathological classification performance. In addition, the image classification model according to an embodiment of the present disclosure may also derive an experimental result showing a performance much similar to or better than that of the radiologist certified by the committee.

The embodiments described above are implemented in a form of a program instruction which may be executed through various computer components and may be recorded in the computer readable recording media. The computer readable recording media may include singly a program instruction, a data file, or a data structure or a combination thereof. The program instruction recorded in the computer readable recording medium may be specially designed and configured for the present invention, or may be publicly known to and used by those skilled in the computer software field. Examples of the computer readable recording media may include a hardware device particularly configured to store and execute program instructions, magnetic media such as hard disks, floppy disks, and magnetic tape, optical recording media such as CD-ROM disks and DVD, magneto-optical media such as floptical disks, ROM, RAM, and flash memories. Examples of the program instructions include a high-level language code executable by a computer by using an interpreter, and the like, as well as a machine language code created by a compiler. The hardware devices may be changed to one or more software modules in order to perform the processing according to the present invention, and an opposite situation thereof is available.

Specific executions described in the present invention are embodiments and the scope of the present invention is not limited even by any method. For brevity of the specification, descriptions of conventional electronic configurations, control systems, software, and other functional aspects of the systems may be omitted. Further, connection or connection members of lines among components exemplarily represent functions connections and/or physical or circuitry connections and may be represented as various functional connections, physical connections, or circuitry connections which are replaceable or added in an actual device. Further, unless otherwise specified, such as “essential”, “important”, etc., the connections may not be components particularly required for application of the present invention.

Some embodiments of the present disclosure provide a method for training an image classification model including a neural network through a computing device, and classifying images having the multi-label nature by label using the trained image classification model, and have an industrial applicability.

What is claimed is:

1. A computer-implemented method comprising:
receiving, by at least one processor of a computer, pathology data comprising medical visual data having a multi-label nature, and storing the pathology data in at least one memory;
executing, by the at least one processor, a diagnostic application stored in the at least one memory, wherein the diagnostic application is configured to derive a pathology-specific determination result for the pathology data using at least one pre-trained artificial intelligence (AI) model accessible via an internal network or an external network;
processing, by the at least one processor, the pathology data using the at least one pre-trained AI model through the diagnostic application, wherein the at least one pre-trained AI model is trained to independently evaluate applicability for each of a plurality of pathologies to the pathology data;
generating, by the at least one processor, pathology judgment data corresponding to each of the plurality of pathologies based on an output of the at least one pre-trained AI model generated by processing the pathology data; and
configuring, by the at least one processor, diagnosis support information including the generated pathology judgment data, and outputting the diagnosis support information via a user interface.

2. The computer-implemented method of claim 1, further comprising:
loading, by the at least one processor, text data including a positive sentence template and a negative sentence template corresponding to each of the plurality of pathologies.

3. The computer-implemented method of claim 2,
wherein the positive sentence template and the negative sentence template are configured as one or more natural language sentences expressing presence or absence of each of the plurality of pathologies.

4. The computer-implemented method of claim 2,
wherein the at least one pre-trained AI model comprises a vision-language model including a vision encoder and a text encoder, and
wherein the processing of the pathology data comprises inputting the pathology data and the text data into the vision-language model.

5. The computer-implemented method of claim 4, further comprising:
generating, by the at least one processor, an image embedding corresponding to the pathology data and a text embedding corresponding to the positive sentence template and the negative sentence template via the vision-language model; and
calculating, by the at least one processor, a similarity between the image embedding and the text embedding.

6. The computer-implemented method of claim 5,
wherein the generating of the pathology judgment data comprises determining the applicability for each of the plurality of pathologies based on a comparison between a similarity with the positive sentence template and a similarity with the negative sentence template.

7. The computer-implemented method of claim 5,
wherein the similarity between the image embedding and the text embedding is calculated based on a cosine similarity or a normalized similarity index.

8. The computer-implemented method of claim 6,
wherein the outputting of the diagnosis support information via the user interface comprises displaying a pathology determined to have a similarity with a positive sentence template equal to or greater than a preset criterion and a pathology determined to have a similarity with a negative sentence template higher than another preset criterion based on the similarity between the image embedding and the text embedding.

9. The computer-implemented method of claim 5,
wherein the outputting of the diagnosis support information via the user interface comprises converting a similarity corresponding to each of the plurality of pathologies into a numerical value or a stepwise indicator and outputting the converted numerical value or the converted stepwise indicator.

10. The computer-implemented method of claim 5,
wherein the outputting of the diagnosis support information via the user interface comprises:
identifying a partial region of the pathology data contributing to generation of the image embedding; and
highlighting the partial region of the pathology data based on the similarity between the image embedding and the text embedding.

11. The computer-implemented method of claim 6, further comprising:
selecting, by the at least one processor, one or more positive sentence templates having a similarity equal to or greater than a preset criterion based on the pathology judgment data.

12. The computer-implemented method of claim 11, further comprising:
generating, by the at least one processor, a natural language diagnostic report corresponding to the pathology data by combining the selected one or more positive sentence templates having the similarity equal to or greater than the preset criterion.

13. The computer-implemented method of claim 6, further comprising:
calculating, by the at least one processor, a confidence score for each of the plurality of pathologies based on a difference between the similarity with the positive sentence template and the similarity with the negative sentence template.

14. The computer-implemented method of claim 13,
wherein the outputting of the diagnosis support information via the user interface comprises displaying the plurality of pathologies in an order of priority based on the confidence score for each of the plurality of pathologies, or identifying and outputting one or more pathologies having a confidence score less than a preset criterion as a subject for re-examination.

15. A system comprising:
at least one memory; and
at least one processor configured to execute instructions stored in the at least one memory to perform operations comprising:
receiving pathology data comprising medical visual data having a multi-label nature and storing the pathology data in the at least one memory;
executing a diagnostic application stored in the at least one memory, wherein the diagnostic application is configured to derive a pathology-specific determination result for the pathology data using at least one pre-trained artificial intelligence (AI) model accessible via an internal network or an external network;
processing the pathology data using the at least one pre-trained AI model through the diagnostic application, wherein the at least one pre-trained AI model is trained to independently evaluate applicability of each for a plurality of pathologies to the pathology data;
generating pathology judgment data corresponding to each of the plurality of pathologies based on an output of the at least one pre-trained AI model generated by processing the pathology data; and
configuring diagnosis support information including the generated pathology judgment data, and outputting the diagnosis support information via a user interface.
